(19) United States
(12) Patent Application Publication
(10) Pub. No.: US 2018/0000454 A1
(43) Pub. Date: Jan. 4, 2018

(54) METHOD AND SYSTEM FOR CONTRAST-AGENT BASED MEDICAL IMAGING

(71) Applicant: Siemens Healthcare GmbH, Erlangen (DE)

(72) Inventors: Matthias SENN, Uttenreuth (DE); Carsten THIERFELDER, Pinzberg (DE); Daniel LERCH, Weilersbach (DE)

(73) Assignee: Siemens Healthcare GmbH, Erlangen (DE)

(21) Appl. No.: 15/623,459

(22) Filed: Jun. 15, 2017

(30) Foreign Application Priority Data
Jun. 30, 2016 (DE) 102016211902.8

(51) Int. Cl. A61B 8/08; A61M 5/00; G01S 7/52; A61B 8/00; G06T 7/00; G01S 15/89; G06F 19/00

(52) U.S. Cl. CPC A61B 8/481; G01S 7/52039; A61M 5/007; A61B 8/461; G01S 7/52036; G01S 15/8906; G06F 19/34; G06T 7/0012

(57) ABSTRACT

A method is for operating a system for contrast-agent based medical imaging including a medical imaging device and a contrast-agent-injection device. In an embodiment, the method includes outputting a first operating element for operating the medical imaging device via a touch-sensitive screen; inputting a first piece of information for operating the medical imaging device via the touch-sensitive screen using the first operating element; outputting a second operating element for operating the contrast-agent-injection device via the touch-sensitive screen; and inputting a second piece of information for operating the contrast-agent-injection device via the touch-sensitive screen using the second operating element.

### PRIORITY STATEMENT

The present application hereby claims priority under 35 U.S.C. §119 to German patent application number DE 102016211902.8 filed Jun. 30, 2016, the entire contents of which are hereby incorporated herein by reference.

### FIELD

At least one embodiment of the invention generally relates to a method for operating a system for contrast-agent based medical imaging, a system for contrast-agent based medical imaging, a computer program product and/or a computer-readable medium.

### BACKGROUND

Contrast-agent based imaging can, for example, be performed within the framework of an examination via computed tomography (CT), magnetic resonance imaging (MR), ultrasound (US), molecular imaging (MI) or single-photon emission computed tomography (SPECT). Here, it is important, on the one hand, to synchronize the imaging and the injection in relation to the temporal sequence and, on the other, in each case to choose the correct parameterization on both the medical imaging device and the contrast-agent-injection device.

Depending upon the imaging modality, the execution of these operational steps can be distributed between different user interfaces. In this case, the user interfaces can be located in different rooms, for example in the examination room and/or in the control room. The examination room contains the medical imaging device and/or the contrast-agent-injection device. The contrast-agent-injection device is often arranged on a truck, which can be moved on wheels, or on a supporting arm suspended from the ceiling.

Typically, an imaging console for operating the medical imaging device and/or a contrast-agent-injection console for operating the contrast-agent-injection device is permanently installed in the control room. Operating elements for operating the contrast-agent-injection device can, for example, be arranged in the form of hardware on the contrast-agent-injection device and/or output via a touch-sensitive screen arranged on the contrast-agent-injection device. Operating elements for operating the medical imaging device can, for example, be arranged in the form of hardware on the medical imaging device and/or output via a touch-sensitive screen arranged on the medical imaging device.

### SUMMARY

At least one embodiment of the invention enables improved operation of a system for contrast-agent based medical imaging.

The claims address embodiments of the invention.

Hereinafter, unless stated otherwise, the term “method” indicates the method for operating a system for contrast-agent based medical imaging. Hereinafter, unless stated otherwise, the term “system” indicates the system for contrast-agent based medical imaging.

The method according to at least one embodiment of the invention for operating a system for contrast-agent based medical imaging comprising a medical imaging device and a contrast-agent-injection device comprises:

outputting a first operating element for operating the medical imaging device via a touch-sensitive screen,

inputting a first piece of information for operating the medical imaging device via the touch-sensitive screen using the first operating element,

outputting a second operating element for operating the contrast-agent-injection device via the touch-sensitive screen, and

inputting a second piece of information for operating the contrast-agent-injection device via the touch-sensitive screen using the second operating element.

One embodiment of the invention provides that a tablet computer comprises the touch-sensitive screen and/or the data processing unit.

The system according to at leas at one embodiment of the invention for contrast-agent based medical imaging comprises:

a medical imaging device,

a contrast-agent-injection device,

a touch-sensitive screen, and

a data processing unit, wherein the data processing unit and/or the system is embodied to carry out a method according to any one of the embodiments disclosed in the description and/or the claims.

The non-transitory computer program product according to at least one embodiment of the invention comprises a computer program, which can be loaded into a memory facility of a data processing unit, with program sections for carrying out all the steps of the method according to any one of the embodiments disclosed in the description and/or in the claims when the computer program is executed by the data processing unit.

The following explains some embodiments with reference to the attached figures. The depiction in the figures is schematic, greatly simplified and not necessarily true to scale.

### BRIEF DESCRIPTION OF THE DRAWINGS

The figures show:

FIG. 1 a flow diagram of a method for operating a system for contrast-agent based medical imaging,

FIG. 2 a further flow diagram of a method for operating a system for contrast-agent based medical imaging,

FIG. 3 a tablet computer with a software application,

FIG. 4 a tablet computer with a first software application and a second software application,

FIG. 5 a system for contrast-agent based medical imaging,

FIG. 6 to FIG. 9 different partial views of the system for contrast-agent based medical imaging.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The method according to at least one embodiment of the invention for operating a system for contrast-agent based medical imaging comprising a medical imaging device and a contrast-agent-injection device comprises:

outputting a first operating element for operating the medical imaging device via a touch-sensitive screen,

inputting a first piece of information for operating the medical imaging device via the touch-sensitive screen using the first operating element,

outputting a second operating element for operating the contrast-agent-injection device via the touch-sensitive screen, and

inputting a second piece of information for operating the contrast-agent-injection device via the touch-sensitive screen using the second operating element.

One embodiment of the invention provides that the method further comprises one or more of:

outputting a third operating element via the touch-sensitive screen, wherein the third operating element comprises a third piece of information relating to a medical examination via the medical imaging device and/or contrast-agent administration via the contrast-agent-injection device, and/or

inputting the first piece of information via a first input command using the first operating element and the third operating element.

One embodiment of the invention provides that the third piece of information is determined based on the second piece of information.

One embodiment of the invention provides that the method further comprises one or more of:

outputting a fourth operating element via the touch-sensitive screen, wherein the fourth operating element comprises a fourth piece of information relating to a medical examination via the medical imaging device and/or contrast-agent administration via the contrast-agent-injection device, and/or

inputting the second piece of information by way of a second input command using the second operating element and the fourth operating element.

One embodiment of the invention provides that the fourth piece of information is determined based on the first piece of information.

One embodiment of the invention provides that the method further comprises:

execution of a software application for operating the medical imaging device and the contrast-agent-injection device comprising the first operating element and the second operating element.

One embodiment of the invention provides that the method further comprises one or more of:

execution of a first software application for operating the medical imaging device comprising the first operating element and/or the fourth operating element, and/or

execution of a second software application for operating the contrast-agent-injection device comprising the second operating element and/or the third operating element.

One embodiment of the invention provides that the method further comprises:

provision of an interface for the transmission of information between the first software application and the second software application.

One embodiment of the invention provides that the interface is based on the client-server model.

One embodiment of the invention provides

that the interface comprises a memory region,

that the first software application has read and/or write access to the memory region, and/or

that the second software application has read and/or write access to the memory region.

One embodiment of the invention provides that the software application is executed by a data processing unit

One embodiment of the invention provides that the first software application and/or the second software application is executed by a data processing unit.

One embodiment of the invention provides that the method further comprises:

transmission of data between the data processing unit and the medical imaging device, wherein the data comprises the first piece of information and/or the fourth piece of information.

One embodiment of the invention provides that the method further comprises:

transmission of data between the data processing unit and the contrast-agent-injection device, wherein the data comprises the second piece of information and/or the third piece of information.

One embodiment of the invention provides that a tablet computer comprises the touch-sensitive screen and/or the data processing unit.

The system according to at leas at one embodiment of the invention for contrast-agent based medical imaging comprises:

a medical imaging device,

a contrast-agent-injection device,

a touch-sensitive screen, and

a data processing unit, wherein the data processing unit and/or the system is embodied to carry out a method according to any one of the embodiments disclosed in the description and/or the claims.

The data processing unit can, in particular, be embodied as part of a data processing system, for example a computer network, a computer, a tablet computer, a smartphone or the like. This data processing system can, for example, be provided by a third party, which is in particular different from the manufacturer of the contrast-agent-injection device and/or the manufacturer of the medical imaging device.

One embodiment of the invention provides that the system further comprises a tablet computer, wherein the tablet computer comprises the touch-sensitive screen and/or the data processing unit.

The non-transitory computer program product according to at least one embodiment of the invention comprises a computer program, which can be loaded into a memory facility of a data processing unit, with program sections for carrying out all the steps of the method according to any one of the embodiments disclosed in the description and/or in the claims when the computer program is executed by the data processing unit.

Program sections that can be read and executed by a data processing unit are stored on the computer-readable medium for carrying out all the steps of the method according to any one of the embodiments disclosed in the description and/or in the claims when the program sections are executed by the data processing unit.

The operation of the medical imaging device can, for example, comprise the selection and/or modification of examination parameters. The operation of the medical imaging device can, for example, comprise the movement of the support plate of the patient support device relative to the gantry of the medical imaging device. The operation of the medical imaging device can, for example, comprise the adjustment of an operating state of the medical imaging device. The adjustment of an operating state of the medical imaging device can, for example, comprise the starting and/or finishing of the examination via the medical imaging device and/or the starting and/or finishing of the acquisition of raw imaging data via the imaging-data-acquisition device.

Information for operating the medical imaging device can, for example, comprise one or more examination parameters and/or the modification of one or more examination parameters. Information for operating the medical imaging device can, for example, comprise a position and/or a change to the position of the support plate relative to the gantry and/or relative to the mounting base. Information for operating the medical imaging device can, for example, comprise a command to start and/or finish the examination via the medical imaging device and/or a command to start and/or finish the acquisition of raw imaging data via the imaging-data-acquisition device.

The examination parameters can, for example, relate to an examination by way of the medical imaging device and/or an examination protocol for an examination by way of the medical imaging device. The examination parameters can, for example, relate to the patient and/or the imaging and/or the contrast-agent administration.

The examination parameters relating to the patient can, in particular, include anatomical data and/or biographical data on the patient, for example the weight and/or age of the patient. The anatomical data on the patient can, in particular, comprise an anatomy determined, for example, based on one or more previous examinations. The examination parameters relating to the imaging can, in particular, comprise the examination region, for example a position and/or an anatomy of the examination region and/or a temporal course of the imaging, for example a starting point and/or a temporal duration of the imaging, and/or data on the radiation, for example the energy and/or dose of the radiation. The examination parameters relating to the contrast-agent administration can, in particular, comprise a designation and/or a quantity of the contrast agent and/or a temporal course of the contrast-agent administration, for example a starting point and/or a temporal duration of the contrast-agent administration.

The operation of the contrast-agent-injection device can, for example, comprise the selection and/or modification of injection parameters. The operation of the contrast-agent-injection device can, for example, comprise the adjustment of an operating state of the contrast-agent-injection device. The adjustment of an operating state of the contrast-agent-injection device can, for example, comprise the starting and/or finishing of the contrast-agent administration via the contrast-agent-injection device.

Information for operating the contrast-agent-injection device can, for example, comprise one or more injection parameters and/or a modification of one or more injection parameters. Information for operating the contrast-agent-injection device can, for example, comprise a command to start and/or finish the contrast-agent administration via the contrast-agent-injection device.

The injection parameters can, for example, relate to contrast-agent administration via the contrast-agent-injection device and/or an injection protocol for contrast-agent administration via the contrast-agent-injection device. The injection parameters can, for example, relate to the patient and/or the imaging and/or the contrast-agent administration.

The injection parameters relating to the patient can, in particular, comprise anatomical and/or biographical data on the patient. The injection parameters relating to the contrast-agent administration can, in particular, comprise a designation and/or a quantity of the contrast agent and/or a temporal course of the contrast-agent administration, for example a starting point and/or a temporal duration of the contrast-agent administration.

Information can be input using an operating element can, for example, be performed in that the information is added in the form of a symbol, in particular text and/or a number, to an input field of the operating element and/or in that the information is selected from a plurality of pieces of information displayed in a option menu of the operating element.

The contrast-agent administration can, for example, be the injection of a first contrast agent, an injection of a second contrast agent, an injection of a solution without contrast agent, in particular a saline solution, or the like or combinations thereof.

The first input command and/or the second input command can, in particular, be a gesture, for example a “drag and drop” gesture.

In particular, the medical imaging device can, on the basis of anatomical and/or biographical data on the patient acquired, for example, in the form of the first piece of information, determine a patient-specific modification of injection parameters and output this in the form of the fourth piece of information.

The software application can, in particular, be a software application installed on the tablet computer with which both the medical imaging device and the contrast-agent-injection device can be controlled. Then, the data exchange between the medical imaging device and the contrast-agent-injection device does not need to take place via two different software applications and can take place by way of one single software application.

In particular, the third piece of information can be determined by way of the second software application based on the second piece of information. In particular, the fourth piece of information can be determined by way of the first software application based on the first piece of information. The first software application and/or the second software application can, in particular, be installed on the tablet computer. The first software application can, for example, be used to perform the operation of the medical imaging device completely and/or at least partially. The second software application can, for example, be used to perform the operation of the contrast-agent-injection device completely and/or at least partially. One embodiment of the invention provides that the software application is embedded in a graphical user interface and/or that the first software application and/or the second software application is embedded in a graphical user interface. The graphical user interface can, for example, be provided by an operating system installed on the tablet computer.

In particular, the interface for the transmission of information between the first software application and the second software application can be embodied in the form of software. The interface can, for example, be used to establish communication between the first software application, which controls the medical imaging device, and the second software application, which controls the contrast-agent-injection device. One embodiment of the invention provides that the interface can be used to transmit information bidirectionally. This, in particular, enables injection parameters and examination parameters to be exchanged between the medical imaging device and the contrast-agent-injection device for documentation purposes.

The first software application can, for example, based on examination parameters present in the first software application, suggest optimized injection parameters and forward these to the second software application.

The second software application can, for example, forward injection parameters actually used by the contrast-agent-injection device for the contrast-agent administration from the contrast-agent-injection device to the first software application. This enables the injection parameters to be archived together with imaging data acquired by the medical imaging device for example in the form of DICOM-data. This, for example, enables information to be transmitted from the contrast-agent-injection device to the medical imaging device and/or documented together with results of the imaging.

The exchange of examination parameters between the first software application and the second software application can also take place by way of the first input command and/or by way of the second input command. The exchange of injection parameters between the first software application and the second software application can also take place by way of the first input command and/or by way of the second input command.

The first software application and/or second software application can, for example, store a file with the information to be transmitted in the memory region of the interface where it can be read by the first software application and/or the second software application. The file form can, for example, be selected as universal thus enabling information also to be transmitted between a first software application and a second software application provided by different manufacturers.

The tablet computer can, for example, comprise a data transmission module embodied to transmit data between the tablet computer and the medical imaging device and/or to transmit data between the tablet computer and the contrast-agent-injection device.

In particular, the tablet computer can establish a data connection to the medical imaging device to transmit the data between the data processing unit and the medical imaging device. In particular, the tablet computer can establish a data connection to the contrast-agent-injection device to transmit the data between the data processing unit and the contrast-agent-injection device. The data connection can, for example, be wireless, in particular based on WLAN, Bluetooth or NFC, and/or wired.

The use of a tablet computer on which the first software application and the second software application are installed in the examination room enables the installation of software for operating the contrast-agent-injection device on the imaging console in the control room to be dispensed with. Software for operating the contrast-agent-injection device, which is provided by third parties and cannot or must not be installed on the imaging console, can often be readily installed on the tablet computer. This simplifies the use of third party-software for operating the contrast-agent-injection device.

The use of a tablet computer on which the first software application and the second software application are installed in the examination room enables a contrast-agent-injection console in the control room to be dispensed with. This both reduces the costs of the contrast-agent-injection device and the labor required for the installation of the contrast-agent-injection device.

The medical imaging device and the contrast-agent-injection device can be connected directly to one another via data connection. To synchronize injection and imaging, it is, for example, possible for a time-critical and/or safety-relevant communication to take place via this direct data connection between the medical imaging device and the contrast-agent-injection device. This direct data connection can, for example, be wired and/or based on a TTL signal.

Alternatively and/or additionally, this direct data connection can comprise a bus system based, for example, on CANopen, TCP/IP or the like. In particular, if an exchange of injection parameters between the medical imaging device and the contrast-agent-injection device is set up by way of the direct data connection and/or by way of the interface, the contrast-agent-injection device can be parameterized and/or controlled via the medical imaging device and hence adapted to the requirements of the user, the demands of the examination and/or the individual features of the patient.

According to one embodiment of the invention, the system for contrast-agent based medical imaging further comprises the following components:
a gantry of a medical imaging device,
a contrast-agent-injection device,
a supporting arm with a frame element, a first connecting element and a second connecting element,
wherein the frame element is connected via the first connecting element to a stationary support frame of the gantry such that at least one part of the frame element is mounted movably relative to the stationary support frame of the gantry,
wherein the contrast-agent-injection device is connected via the second connecting element to the at least one part of the frame element such that the contrast-agent-injection device is mounted movably relative to the at least one part of the frame element.

One embodiment of the invention provides

that the second connecting element comprises a first connecting region in which the contrast-agent-injection device can be connected to the second connecting element and/or

that the second connecting element comprises a second connecting region in which an additional component can be connected to the second connecting element when the contrast-agent-injection device in the first connecting region is connected to the second connecting element.

One embodiment of the invention provides

that the frame element by way of the first connecting element is connected to the stationary support frame of the gantry such that the frame element is mounted rotatably about a first rotary axis and/or tiltably about a first tilt axis relative to the stationary support frame of the gantry,

that the contrast-agent-injection device is connected via the second connecting element to the frame element such that the contrast-agent-injection device is mounted rotatably about a second rotary axis and/or tiltably about a second tilt axis relative to the frame element and/or

that the additional component is connected via the second connecting element to the frame element such that the additional component is mounted rotatably about the second rotary axis and/or tiltably about the second tilt axis and/or a third tilt axis relative to the frame element.

One embodiment of the invention provides

that the contrast-agent-injection device can be connected to the second connecting element in a positive and/or detachable manner and/or

that the additional component can be connected to the second connecting element in a positive and/or detachable manner.

One embodiment of the invention provides that the first connecting region and the second connecting region are arranged symmetrically in relation to the second rotary axis.

One embodiment of the invention provides that the additional component comprises a touch-sensitive screen and/or a tablet computer.

One embodiment of the invention provides that the support frame comprises a hollow space that extends along the support frame such that a cable can be arranged in the hollow space, wherein said cable extends within the hollow space from the first connecting element to the second connecting element.

One embodiment of the invention provides that the system further comprises a cable, which extends within the hollow space from an internal space of the stationary support frame of the gantry as far as the contrast-agent-injection device and/or as far as the additional component.

One embodiment of the invention provides

that the first connecting element comprises a first support region, wherein the frame element is mounted via the first support region rotatably about a first rotary axis relative to the stationary support frame of the gantry and/or

that the first connecting element comprises a second support region, wherein the frame element is mounted via the second support region tiltably about a first tilt axis relative to the stationary support frame of the gantry.

One embodiment of the invention provides

that, in the first support region, the first connecting element comprises a rotary-motion-restricting device for restricting the rotary motion of the frame element about the first rotary axis to a specific rotary angle range and/or

that, in the second support region, the first connecting element comprises a tilting-motion-restricting device for restricting the tilting motion of the frame element about the first tilt axis to a specific tilt angle range.

One embodiment of the invention provides

that the rotary-motion-restricting device comprises a groove in the form of a circular arc and a pin guided in the groove,

that the rotary motion of the frame element about the first rotary axis is coupled to a motion of the pin along the groove and restricted to a rotary angle range encompassed by the groove.

One embodiment of the invention provides

that, in the first support region, the first connecting element comprises a rotary-angle-stabilization device for stabilizing a rotary angle of the frame element in relation to a rotary motion of the frame element about the first rotary axis and/or

that, in the second support region, the first connecting element comprises a tilt-angle-stabilizing device for stabilizing a tilt angle of the frame element in relation to a tilting motion of the frame element about the first tilt axis.

One embodiment of the invention provides

that the rotary-angle-stabilization device comprises at least one friction contact,

that the rotary motion of the frame element about the first rotary axis is friction-coupled to the at least one friction contact of the rotary-angle-stabilization device,

that the rotary angle can be stabilized via the static friction on the at least one friction contact of the rotary-angle-stabilization device.

One embodiment of the invention provides

that the tilt-angle-stabilizing device comprises at least one friction contact,

that the tilting motion of the frame element about the first tilt axis is friction-coupled to the at least one friction contact of the tilt-angle-stabilizing device,

that the tilt angle can be stabilized via the static friction on the at least one friction contact of the tilt-angle-stabilizing device.

One embodiment of the invention provides

that at least one friction partner of the at least one friction contact of the rotary-angle-stabilization device is arranged in a ring shape and/or in a circular arc shape about the first rotary axis and/or is made of plastic and/or

that at least one friction partner of the at least one friction contact of the tilt-angle-stabilizing device is arranged in a ring shape and/or in a circular arc shape about the first tilt axis and/or is made of plastic.

One embodiment of the invention provides

that the rotary-angle-stabilization device comprises a clamping element, which interacts with the at least one friction contact of the rotary-angle-stabilization device such that the contact pressure underlying the friction on the at least one friction contact of the rotary-angle-stabilization device is induced at least partially by mechanically stressing the clamping element of the rotary-angle-stabilization device and/or

that the tilt-angle-stabilizing device comprises a clamping element, which interacts with the at least one friction contact of the tilt-angle-stabilizing device such that the contact pressure underlying the friction on the at least one friction contact of the tilt-angle-stabilizing device is induced at least partially by mechanically stressing the clamping element of the tilt-angle-stabilizing device.

One embodiment of the invention provides

that the clamping element of the rotary-angle-stabilization device comprises a spring and/or wherein the spring of the clamping element of the rotary-angle-stabilization device comprises one or more windings arranged about the first rotary axis and/or

that the clamping element of the tilt-angle-stabilizing device comprises a spring and/or wherein the spring of the clamping element of the tilt-angle-stabilizing device comprises one or more windings arranged about the first tilt axis.

One embodiment of the invention provides

that the first rotary axis is vertical and/or the first tilt axis is horizontal.

One embodiment of the invention provides

that, at least for a tilt angle of the frame element in relation to a tilting motion of the frame element about the first tilt axis, the second rotary axis is vertical and/or the second tilt axis is horizontal.

The use of the support arm according to any one of the embodiments disclosed in the description and/or in the claims enables both an arrangement of the support arm on the ceiling of the examination room and an arrangement of the support arm on the floor of the examination room to be dispensed with. This reduces the space required and the demands on the structural features of the examination room. In particular, the frame element can be arranged between the first connecting element and the second connecting element.

The tiltable mounting about the first tilt axis and/or about the second tilt axis enables the adaptation of the height of the contrast-agent-injection device for example in dependence on the user's requirements and/or the demands of the examination. Users can, for example, drive the tilting motion manually themselves and thus bring the supporting arm into the desired position.

In particular as an alternative to the additional component being mounted tiltably about the second tilt axis, the additional component can be mounted about a third tilt axis. In particular, the third tilt axis can be parallel to the second tilt axis. In particular, the distance between the second tilt axis and the third tilt axis can be smaller than, for example, a diagonal of the touch-sensitive screen or, for example, a fifth of the diagonal of the touch-sensitive screen.

In particular, in the first connecting region, the second connecting element can be embodied for positive and/or detachable coupling and decoupling of the contrast-agent-injection device. In particular, in the second connecting region, the second connecting element can be embodied for positive and/or detachable coupling and decoupling of the additional component. The coupling and decoupling of the contrast-agent-injection device and/or the additional component can, for example, be performed manually by the user.

Alternatively or additionally, the additional component can comprise one or more intervention tools. In particular, the additional component can be a tablet computer according to any one of the embodiments disclosed in the description and/or in the claims. In particular, the tablet computer can be embodied for operating the medical imaging device and/or for operating the contrast-agent-injection device.

One embodiment of the invention provides that a normal vector of a screen area of the touch-sensitive screen is substantially perpendicular to the second tilt axis. One embodiment of the invention provides that the contrast-agent-injection device comprises a flat input and/or display region and that a normal vector of the flat input and/or display region of the contrast-agent-injection device is substantially perpendicular to the second tilt axis. The flat input and/or display region of the contrast-agent-injection device can, for example, be integrated in an enclosure of the contrast-agent-injection device.

One embodiment of the invention provides that the normal vector the screen area of the touch-sensitive screen, which points away from the touch-sensitive screen, is substantially parallel to the normal vector of the flat input and/or display region of the contrast-agent-injection device pointing away from the contrast-agent-injection device. The contrast-agent-injection device and the tablet computer can hence be arranged in a clearly visible manner next to each other on the second connecting element. The better visibility enables error sources to be eliminated during the operation of the medical imaging device and/or the contrast-agent-injection device. In particular, a rotary motion of the frame element about the first rotary axis enables the contrast-agent-injection device and the tablet computer to be available both on the front side of the medical imaging device, in which the patient support device is located, and on the rear side of the medical imaging device. This can prevent the user having to travel unnecessary distances.

The frame element can, for example, be formed by a steel tube and/or a plastic enclosure. The supporting arm, in particular the frame element, can, for example, be coated with paint. In particular, the paint can enable the color to be adapted to the gantry and/or demands relating to electrostatic safety to be achieved.

The cable can, for example, comprise one or more lines for supplying energy to the contrast-agent-injection device and/or the additional component and/or one or more lines for data transmission between the medical imaging device on the one hand and the contrast-agent-injection device and/or the additional component on the other.

The stationary support frame of the gantry can, for example, be embodied in the region of the first support region for receiving the first connecting element and/or for receiving the forces and/or torques acting on the supporting arm. For example, in the region of the first support region, a receiving unit provided by the enclosure of the gantry and/or integrated in the enclosure of the gantry can be arranged to receive the first connecting element.

The groove and the pin can restrict the rotary motion to a specific rotary angle range. In particular, this can avoid a collision of the support arm and/or the contrast-agent-injection device with the patient and/or with a part of the medical imaging device, for example the gantry.

One embodiment of the invention provides that the second connecting element comprises a first rotary-angle-stabilization device for stabilizing a rotary angle of the contrast-agent-injection device in relation to a rotary motion of the contrast-agent-injection device about the second rotary axis comprises and/or

that the second connecting element comprises a first tilt-angle-stabilizing device for stabilizing a tilt angle of the contrast-agent-injection device in relation to a tilting motion of the contrast-agent-injection device about the second tilt axis. One embodiment of the invention provides that the second connecting element comprises a second rotary-angle-stabilization device for stabilizing a rotary angle of the additional component in relation to a rotary motion of the contrast-agent-injection device about the second rotary axis comprises and/or

that the second connecting element comprises a second tilt-angle-stabilizing device for stabilizing a tilt angle of the additional component in relation to a tilting motion of the additional component about the second tilt axis.

The first rotary-angle-stabilization device of the second connecting element, the second rotary-angle-stabilization device of the second connecting element, the first tilt-angle-stabilizing device of the second connecting element and/or the second tilt-angle-stabilizing device of the second connecting element can each be developed with at least one friction contact and/or with a clamping element, as described with reference to the example of the rotary-angle-stabilization device of the first connecting element and/or the example of the tilt-angle-stabilizing device of the first connecting element.

In particular, the friction partner of the at least one friction contact of the rotary-angle-stabilization device can be selected and arranged such that the friction on the at least one first friction contact is, on the one hand, sufficiently strong to ensure that the forces and/or torques induced by the mass and/or the operation of the contrast-agent-injection device and/or the additional component do not result in a rotary motion of the frame element about the first rotary axis and, on the other, a rotary motion of the frame element about the first rotary axis can be driven manually by a user by applying muscle power to the supporting arm.

In particular, the friction partner of the at least one friction contact of the tilt-angle-stabilizing device can be selected and arranged such that the friction on the friction contact of the tilt-angle-stabilizing device is, on the one hand, sufficiently strong to ensure that the forces and/or torques induced by the mass and/or the operation of the contrast-agent-injection device and/or the additional do not result in a tilting motion of the frame element about the first tilt axis and, on the other, a tilting motion of the frame element about the first tilt axis can be driven manually by a user by applying muscle power to the supporting arm.

In particular, the at least one friction partner of the at least one friction contact of the rotary-angle-stabilization device can comprise a region made of plastic and/or have a plastic surface. In particular, the at least one friction partner of the at least one friction contact of the tilt-angle-stabilizing device can comprise a region made of plastic and/or have a plastic surface. The plastic can, for example, be polyamide (PA) and/or based on polyamide (PA).

One embodiment of the invention provides that the data processing unit is formed at least partially by a data processing system and/or that one or more components of the data processing unit is formed at least partially by a data processing system.

The data processing system can, for example, comprise one or more components in the form of hardware and/or one or more components in the form of software. The data processing system can, for example, be formed at least partially by a cloud computing system. The data processing system can, for example, be or comprise a cloud computing system, a computer network, a computer, a tablet computer, a smartphone or the like or combinations thereof. The hardware can, for example, interact with software and/or be configurable by way of software. The software can, for example, be executed via the hardware. The hardware can, for example, be a storage system, an FPGA system (field-programmable gate array), an ASIC system (application-specific integrated circuit), a microcontroller system, a processor system and combinations thereof. The processor system can, for example, comprise a microprocessor and/or a plurality of interacting microprocessors.

A data transfer between components of the data processing system can, for example, in each case take place by way of a suitable data transfer-interface. The data transfer-interface to transfer data transfer to and/or from a component of the data processing system can be implemented at least partially in the form of software and/or at least partially in the form of hardware. The data transfer-interface can, for example, be embodied to store data in and/or to load data from a region of the storage system, wherein one or more components of the data processing system can access this region of the storage system.

The computer program can, for example, be loadable into the storage system of the data processing system and executable by the processor system of the data processing system. The data processing system can, for example, be embodied by way of the computer program such that the data processing system is able to carry out the steps of a method according to any one of the embodiments disclosed in the description and/or in the claims when the computer program is executed by the data processing system.

The computer program product can, for example, be the computer program or comprise at least one additional component in addition to the computer program. The at least one additional component of the computer program product can be embodied as hardware and/or as software. The computer program product can, for example, comprise a storage medium on which at least one part of the computer program product is stored and/or a key for authenticating a user of the computer program product, in particular in the form of a dongle. The computer program product and/or the computer program can, for example, comprise a cloud application program embodied to distribute program sections of the computer program between different processing units, in particular different computers, of a cloud computing system, wherein each of the processing units is embodied to execute one or more program sections of the computer program.

The computer-readable medium can, for example, store the computer program product according to any one of the embodiments disclosed in the description and/or in the claims and/or the computer program according to any one of the embodiments disclosed in the description and/or in the claims. The computer-readable medium can, for example, be a memory stick, a hard disk or another kind of data medium, which can in particular be connected detachably to the data processing system or permanently integrated in the data processing system. The computer-readable medium can, for example, form at least one region of the storage system of the data processing system.

The medical imaging device can, for example, be selected from the imaging modality group consisting of an X-ray device, a C-arm X-ray device, a computed tomography device (CT device), a molecular imaging device (MI device), a single photon emission computed tomography device (SPECT device), a positron emission tomography device (PET device), a magnetic resonance imaging (MRT device) and combinations thereof (in particular PET-CT device, PET-MR device). The medical imaging device can further comprise a combination of an imaging modality selected, for example, from the imaging modality group and an irradiation modality. In this case, the irradiation modality can, for example, comprise an irradiation unit for therapeutic irradiation. Without restricting the general concept of the invention, with some of the embodiments a computed tomography device is described by way of example as a medical imaging device.

Within the framework of the invention, features, which are described in relation to different embodiments and/or different categories (method, device, system, etc.) can be combined to form further embodiments of the invention. In particular, the substantive claims can also be developed with the features described or claimed in connection with a method. In this case, a functional feature of a method can be implemented by way of a correspondingly embodied substantive component. In addition to the embodiments of the invention expressly described in this application, numerous further embodiments of the invention are conceivable which can be achieved by the person skilled in the art without departing from the scope of the invention as specified in the claims.

The use of the indefinite article “a” or “an” does not preclude the possibility that the feature in question may also be present on a multiple basis. The use of the expression “comprise” does not preclude the possibility that terms linked by way of the term “comprise” may be identical. For example, the medical imaging device comprises the medical imaging device. The use of the term “unit” does not preclude the possibility that the subject matter to which the term “unit” relates may comprise a plurality of components that are spatially separate from one another.

In the context of the present application, the use of ordinal numbers (first, second, third etc.) in the designation of features is used above all for better differentiation of features designated using ordinal numbers. The absence of a feature designated by a combination of a given ordinal number and a term does not preclude the possibility that a feature may be present that is designated by a combination of an ordinal following the given ordinal and the term.

In the context of the present application, the term “based on” can in particular be understood as having the meaning of the term “using”. In particular, wording according to which a first feature is generated (alternatively: ascertained, determined, etc.) based on a second feature does not preclude the possibility that the first feature may be generated (alternatively: ascertained, determined, etc.) based on a third feature.

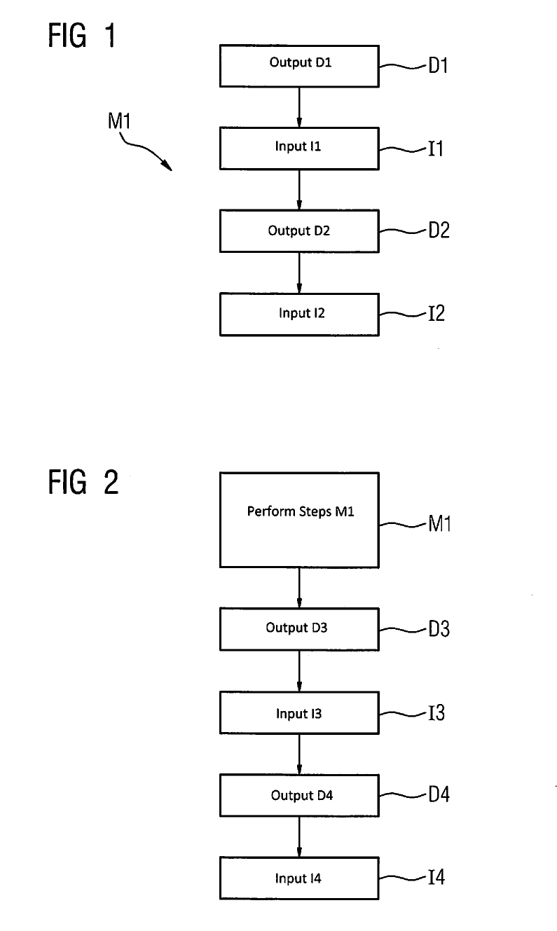

FIG. 1 shows a flow diagram of a method for operating a system 1 for contrast-agent based medical imaging comprising a medical imaging device 2 and a contrast-agent-injection device ID.

The embodiment shown in FIG. 1 provides that the method comprises the following steps:

outputting D1 a first operating element E1 for operating the medical imaging device 2 via a touch-sensitive screen TS,

inputting I1 a first piece of information for operating the medical imaging device 2 via the touch-sensitive screen TS using the first operating element E1,

outputting D2 a second operating element E2 for operating the contrast-agent-injection device ID via the touch-sensitive screen TS,

inputting I2 a second piece of information for operating the contrast-agent-injection device ID via the touch-sensitive screen TS using the second operating element E2.

FIG. 2 shows a further flow diagram of a method for operating a system 1 for contrast-agent based medical imaging comprising a medical imaging device 2 and a contrast-agent-injection device ID.

The embodiment shown in FIG. 2 provides that, in addition to the steps shown in FIG. 1, which are depicted in simplified form in FIG. 2, the group of steps M1, further comprises the following steps:

outputting D3 a third operating element E3 via the touch-sensitive screen TS, wherein the third operating element E3 comprises a third piece of information relating to a medical examination via the medical imaging device 2 and/or contrast-agent administration via the contrast-agent-injection device ID,

inputting 13 the first piece of information via a first input command G1 using the first operating element E1 and the third operating element E3,

outputting D4 a fourth operating element E4 via the touch-sensitive screen TS, wherein the fourth operating element E4 comprises a fourth piece of information relating to a medical examination via the medical imaging device 2 and/or contrast-agent administration via the contrast-agent-injection device ID,

inputting 14 the second piece of information via a second input command G2 using the second operating element E2 and the fourth operating element E4.

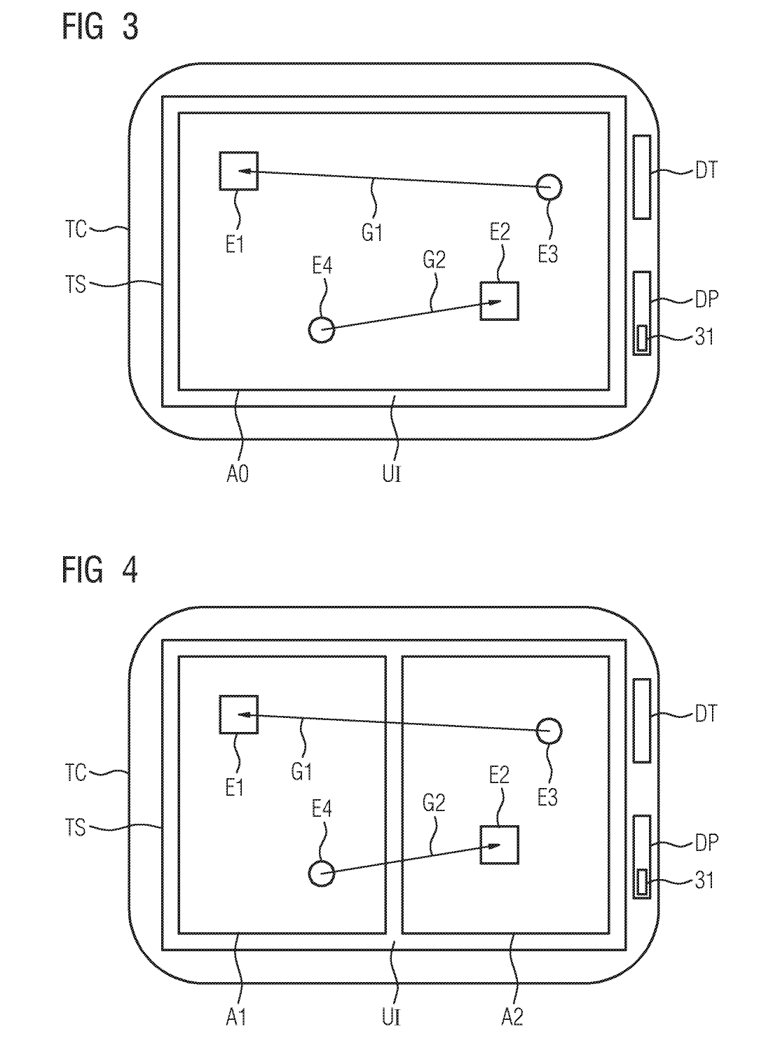

FIG. 3 shows a tablet computer TC with a software application A0 for operating the medical imaging device 2 and the contrast-agent-injection device ID.

The embodiment shown in FIG. 3 provides

that the tablet computer TC comprises the touch-sensitive screen TS, the data processing unit DP and the data transmission module and

that the software application A0 which is executed by the data processing unit DP is installed on the tablet computer TC. The data processing unit DP comprises the storage facility 31 in the form of a computer-readable medium. The data transmission module is embodied to transmit data between the tablet computer TC and the medical imaging device 2 and to transmit data between the tablet computer TC and the contrast-agent-injection device ID. Also installed on the tablet computer TC is an operating system provided by the graphical user interface UI. The software application A0 is embedded in the graphical user interface UI. The software application A0 comprises the first operating element E1, the second operating element E2, the third operating element E3 and the fourth operating element E4.

The first input command G1 is a gesture comprising the following steps:

marking the third operating element E3,

pulling the third operating element E3 to the first operating element E1,

placing the third operating element E3 on the first operating element E1.

The second input command G2 is a gesture comprising the following steps:

marking the fourth operating element E4,

pulling the fourth operating element E4 to the second operating element E2,

placing the fourth operating element E4 on the second operating element E2.

Gestures of this kind are also known to the person skilled in the art by the term “drag and drop” gesture.

FIG. 4 shows a tablet computer TC with a first software application A1 for operating the medical imaging device 2 and a second software application A2 for operating the contrast-agent-injection device ID.

The embodiment shown in FIG. 4 provides that the first software application A1 and the second software application A2, which are each executed by the data processing unit DP, are installed on the tablet computer TC. Also installed on the tablet computer TC is an operating system that provides the graphical user interface UI. The first software application A1 and the second software application A2 are each embedded in the graphical user interface UI. The first software application A1 comprises the first operating element E1 and the fourth operating element E4. The second software application A2 comprises the third operating element E3 and the second operating element E2.

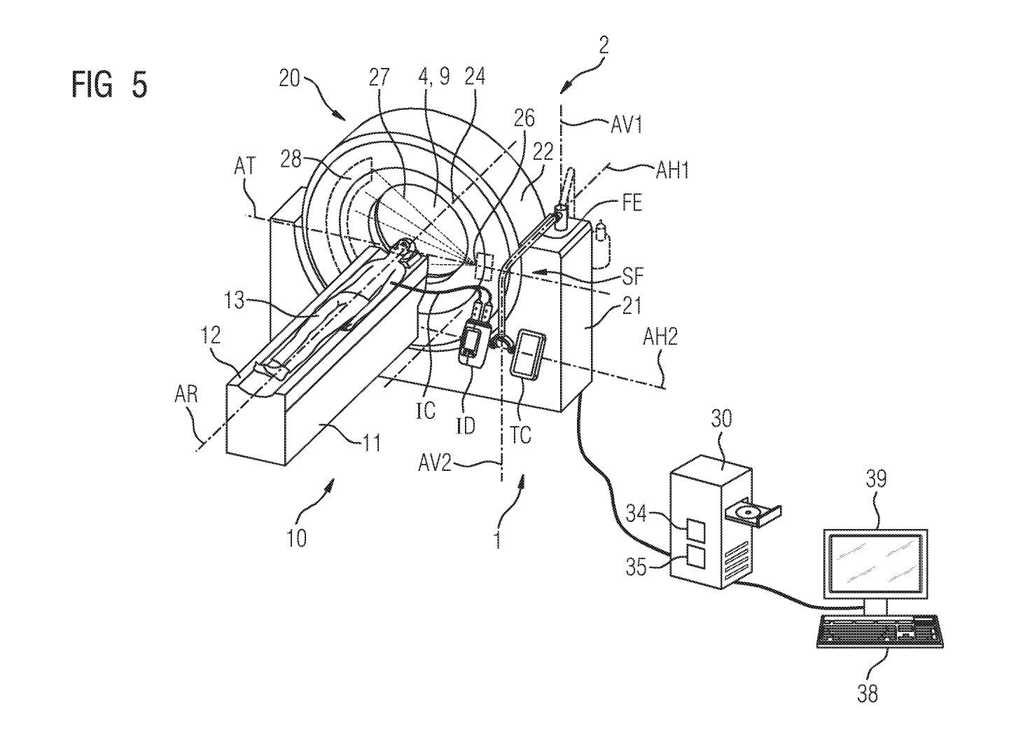

FIG. 5 shows a system 1 for contrast-agent based medical imaging comprising a medical imaging device 2.

Without restricting the general concept of the embodiments of the invention, the medical imaging device 2 shown is by way of example a computed tomography device. The medical imaging device 2 comprises the gantry 20, the tunnel-shaped opening 9, the patient support device 10 and the control device 30.

The gantry 20 comprises the stationary support frame 21, the tilt frame 22 and the rotor 24. The tilt frame 22 is arranged via a tilt support device on the stationary support frame 21 so as to be tiltable about a gantry tilt axis AT relative to the stationary support frame 21. The rotor 24 is arranged via a rotary support device on the tilt frame 22 so as to be rotatable about a gantry rotation axis relative to the tilt frame 22. The gantry tilt axis AT is perpendicular to the system axis AR. The system axis AR and the gantry tilt axis AT are located in a horizontal plane. The gantry rotation axis is perpendicular to the gantry tilt axis AT and substantially parallel to the system axis AR. The patient 13 can be introduced into the tunnel-shaped opening 9. The tunnel-shaped opening 9 contains the acquisition region 4. A region of the patient 13 to be imaged can be positioned in the acquisition region 4 such that the radiation 27 can travel from the radiation source 26 to the region to be imaged and, following interaction with the region to be imaged, travel to the radiation detector 28.

The patient support device 10 comprises the mounting base 11 and the support plate 12 for mounting the patient 13. The support plate 12 is arranged relative to the mounting base 11 movably on the mounting base 11 such that the support plate 12 can be introduced into the acquisition region 4 of a longitudinal direction of the support plate 12, in particular along the system axis AR.

The medical imaging device 2 is embodied to acquire imaging data based on electromagnetic radiation 27. The medical imaging device 2 comprises an imaging data-acquisition unit. The imaging data-acquisition unit is a projection-data-acquisition unit with the radiation source 26, for example an X-ray source, and the detector 28, for example a X-ray detector, in particular an energy-resolving X-ray detector. The radiation source 26 is arranged on the rotor 24 and embodied to emit radiation 27, for example X-rays, with radiation quanta 27. The detector 28 is arranged on the rotor 24 and embodied to detect the radiation quanta 27. The radiation quanta 27 can travel from the radiation source 26 to the region of the patient 13 to be imaged and, following interaction with the region to be imaged, arrive at the detector 28. This enables the imaging data-acquisition unit to acquire imaging data of the region to be depicted in the form of projection-data.

The control device 30 is embodied to receive the imaging data acquired by the imaging data-acquisition unit. The control device 30 is embodied to control the medical imaging device 2. The control device 30 comprises the image-reconstruction facility 34. The image-reconstruction facility 34 can reconstruct a medical image data set based on the imaging data.

The medical imaging device 2 comprises an input device 38 and an output device 39 each of which is connected to the control device 30. The input device 38 is embodied to input control information, for example image reconstruction parameters, examination parameters or the like. The output device 39 is in particular embodied to output control information, images and/or tones.

The embodiment shown in FIG. 5 provides that the system 1 comprises the following components for contrast-agent based medical imaging:

a gantry 20 of a medical imaging device,

a contrast-agent-injection device ID,

a supporting arm SF with a frame element FE, a first connecting element C1 and a second connecting element C2,

wherein the frame element FE is connected via the first connecting element C1 to a stationary support frame 21 the gantry 20 such that at least one part of the frame element FE is mounted movably relative to the stationary support frame 21 the gantry 20,

wherein the contrast-agent-injection device ID is connected via the second connecting element C2 to the at least one part of the frame element FE such that the contrast-agent-injection device ID is mounted movably relative to the at least one part of the frame element FE.

The embodiment shown in FIG. 5 further provides

that the second connecting element C2 comprises a first connecting region R1 in which the contrast-agent-injection device ID can be connected to the second connecting element C2,

that the second connecting element C2 comprises a second connecting region R2 in which an additional component can be connected to the second connecting element C2, while the contrast-agent-injection device ID in the first connecting region R1 is connected to the second connecting element C2,

that the frame element FE is connected via the first connecting element C1 to the stationary support frame 21 the gantry 20 such that the frame element FE is mounted relative to the stationary support frame 21 of the gantry 20 rotatably about a first rotary axis AV1 and tiltably about a first tilt axis AH1,

that the contrast-agent-injection device ID is connected via the second connecting element C2 to the frame element FE such that the contrast-agent-injection device ID is mounted relative to the frame element FE rotatably about a second rotary axis AV2 and tiltably about a second tilt axis AH2,

that the additional component is connected via the second connecting element C2 to the frame element FE such that the additional component is mounted relative to the frame element FE rotatably about the second rotary axis AV2 and tiltably about the second tilt axis AH2 and

that the additional component is a tablet computer TC.

The embodiment shown in FIG. 5 further provides that the first rotary axis AV1 is vertical and the first tilt axis AH1 is horizontal. At least for the tilt angle of the frame element FE in relation to a tilting motion of the frame element FE about the first tilt axis AH1 shown in FIG. 5, the second rotary axis AV2 is vertical and the second tilt axis AH2 is horizontal.

The first connecting element C1 is arranged in a region of a tilt support of the tilt support device the gantry 20. The first rotary axis AV1 extends through the region of the tilt support of the tilt support device.

In the operating state of the system 1 depicted in FIG. 5, the frame element FE is rotated about the first rotary axis AV1 such that the second connecting element C2, the contrast-agent-injection device ID and the tablet computer TC are located on the front side of the medical imaging device 2 in which the patient support device 10 is also located. In FIG. 5, dashed lines indicate a further operating state of the system 1 in which the frame element FE is arranged about the first rotary axis AV1 such that the second connecting element C2, the contrast-agent-injection device ID and the tablet computer TC are located on the rear side of the medical imaging device 2. The gantry 20 of the medical imaging device 2 is located between the front side of the medical imaging device 2 and the rear side of the medical imaging device 2.

The control device 30 comprises the data transmission unit DT 35. The data transmission unit DT 35 is embodied to transmit data between the control device 30 and the tablet computer TC and to transmit data between the control device 30 and the contrast-agent-injection device ID. The contrast-agent-injection device ID comprises the data transmission unit DT 36. The data transmission unit DT 36 is embodied to transmit data between the contrast-agent-injection device ID and the tablet computer TC and to transmit data between the contrast-agent-injection device ID and the control device 30.

The contrast-agent-injection device ID is connected to the patient 13 via the injection line IC. The contrast-agent-injection device ID comprises a first injector IN1 and a second injector IN2. The first injector IN1 can, for example, inject a first contrast agent into the patient 13. The second injector IN2 can, for example, inject a second contrast agent, and/or a solution without contrast agent, in particular a saline solution, into the patient 13.

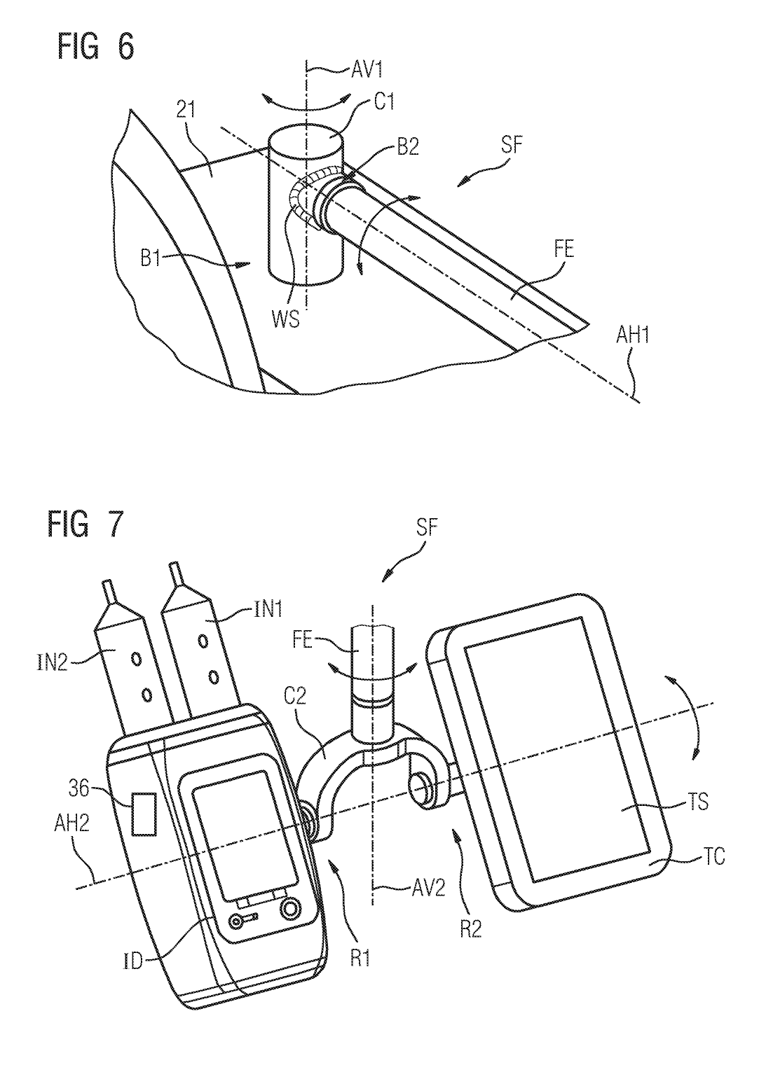

The embodiment shown in FIG. 6 provides

that the first connecting element C1 comprises a first support region B1, wherein the frame element FE is mounted via the first support region B1 relative to the stationary support frame 21 of the gantry 20 rotatably about a first rotary axis AV1 and

that the first connecting element C1 comprises a second support region B2, wherein the frame element FE is mounted via the second support region B2 relative to the stationary support frame 21 of the gantry 20 tiltably about a first tilt axis AH1.

The embodiment shown in FIG. 7 provides

that the first connecting region R1 and the second connecting region R2 are arranged symmetrically in relation to the second rotary axis AV2.

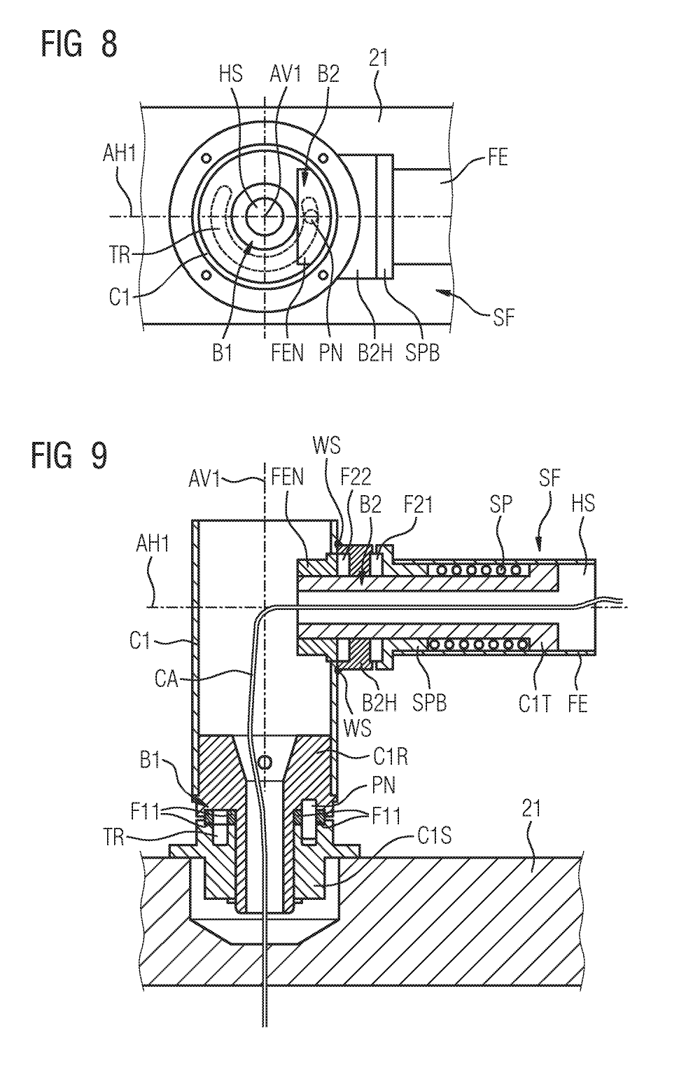

The embodiment shown in FIG. 8 provides

that, in the first support region B1, the first connecting element C1 comprises a rotary-motion-restricting device and

that the rotary-motion-restricting device comprises a groove TR in the form of a circular arc and a pin PN guided in the groove TR.

The embodiment shown in FIG. 9 provides

that the support frame comprises a hollow space HS,

that the system 1 comprises a cable CA, which extends within the hollow space HS from an internal space of the stationary support frame 21 of the gantry 20 as far as the contrast-agent-injection device ID and as far as the tablet computer TC,

that, in the first support region B1, the first connecting element C1 comprises a rotary-angle-stabilization device, and

that, in the second support region B2, the first connecting element C1 comprises a tilt-angle-stabilizing device.

The rotary-angle-stabilization device comprises at least one friction contact. The at least one friction contact of the rotary-angle-stabilization device is formed by the ring-shaped disk F11 with the part C1S of the first connecting element C1 and/or with the part C1R of the first connecting element C1.

The part C1S of the first connecting element C1 is permanently arranged on the stationary support frame 21 the gantry 20. The part C1R of the first connecting element C1 is mounted via the first support region B1 relative to the stationary support frame 21 of the gantry 20 rotatably about the first rotary axis AV1 and connected via the second support region B2 to the frame element FE.

The ring-shaped disk F11 forms a friction partner of the at least one friction contact of the rotary-angle-stabilization device, is arranged in a ring shape about the first rotary axis AV1 and is made of plastic. Optionally, the ring-shaped disk F11 can be connected, for example bonded or connected in a positively fitting manner, either to the part C1S of the first connecting element C1 or to the part C1R of the first connecting element C1 in particular permanently arranged in relation to a rotational motion about the first rotary axis AV1. The ring-shaped disk F11 comprises a circular-arc-shaped hole corresponding to the groove TR. The pin PN extends through the circular-arc-shaped hole of the ring-shaped disk F11.

Alternatively to the ring-shaped disk F11, which comprises a circular-arc-shaped hole, it is possible to use two or more ring-shaped disks arranged concentrically about the first rotary axis AV1. In particular, a ring-shaped gap can be located between two ring-shaped disks arranged concentrically about the first rotary axis AV1, wherein a circular-arc-shaped section of the ring-shaped gap corresponds to the groove TR. Hence, the pin PN can extend through the circular-arc-shaped section of the ring-shaped gap.

The tilt-angle-stabilizing device comprises a first friction contact and a second friction contact.

The first friction contact of the tilt-angle-stabilizing device is formed by the ring-shaped disk F21 with the part C1T of the first connecting element C1 and/or with the piston element SPB.

The ring-shaped disk F21 forms a friction partner of the first friction contact of the tilt-angle-stabilizing device, is arranged in a ring shape about the first tilt axis AH1 and made of plastic. Optionally, the ring-shaped disk F21 can be connected, for example bonded or connected in a positively fitting manner, either to the part C1T of the first connecting element C1 or to the piston element SPB in particular permanently arranged in relation to a rotational motion about the first tilt axis AH1.

The second friction contact of the tilt-angle-stabilizing device is formed by the ring-shaped disk F22 with the part C1T of the first connecting element C1 and/or with the part C1R of the first connecting element C1. The part C1T of the first connecting element C1 is permanently arranged on the frame element FE.

The ring-shaped disk F22 forms a friction partner of the second friction contact of the tilt-angle-stabilizing device, is arranged in a ring shape about the first tilt axis AH1 and is made of plastic. Optionally, the ring-shaped disk F22 can be connected, for example bonded or connected in a positively fitting manner, either to the part C1T of the first connecting element C1 or to the part C1R of the first connecting element C1 in particular permanently arranged in relation to a rotational motion about the first tilt axis AH1.

The tilt-angle-stabilizing device comprises a clamping element SP in the form of a spring the windings of which are arranged about the first tilt axis AH1. The piston element SPB is arranged on a first end of the clamping element SP. The clamping element SP is arranged with a second end of the clamping element SP on the part C1T of the first connecting element C1.

The second support region B2 comprises a bearing housing B2H. The bearing housing B2H is connected via the welded seam WS to the part C1R of the first connecting element C1. The bearing housing B2H is embodied to receive forces and torques acting on the frame element FE and forward them to the part C1R of the first connecting element C1. The bearing housing B2H comprises a bore hole. The part C1T of the first connecting element C1 extends through the bore hole of the bearing housing B2H. On the side facing away from the piston element FEN in relation to the bearing housing B2H, the part C1T of the first connecting element C1 is fixed with a fastening element relative to the bearing housing B2H. The fastening element is a nut FEN screwed to a thread of the first part C1T of the first connecting element C1. The bearing housing B2H, the ring-shaped disk F21 and the ring-shaped disk F22 are located between the nut FEN and the piston element SPB and pressed together by the nut FEN and the piston element SPB.

The clamping element SP generates a mechanical stress, in particular a strain-stress, with which the first end of the clamping element SP and the second end of the clamping element SP are pressed apart. This causes the piston element SPB to be pressed onto the ring-shaped disk F21 and the nut FEN onto the ring-shaped disk F22.

At least in relation to the tilting motion of the frame element FE about the first tilt axis AH1, the part C1T of the first connecting element C1 is permanently connected to the frame element FE. The embodiment shown in FIG. 9 provides that the part C1T of the first connecting element C1, the clamping element SP, the piston element SPB, the ring-shaped disk F21 and the ring-shaped disk F22 together follow the tilting motion of the frame element FE about the first tilt axis AH1.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for operating a system for contrast-agent based medical imaging including a medical imaging device and a contrast-agent-injection device, the method comprising:
outputting a first operating element for operating the medical imaging device via a touch-sensitive screen;
inputting a first piece of information for operating the medical imaging device via the touch-sensitive screen using the first operating element;
outputting a second operating element for operating the contrast-agent-injection device via the touch-sensitive screen; and
inputting a second piece of information for operating the contrast-agent-injection device via the touch-sensitive screen using the second operating element.

2. The method of claim 1, further comprising:
outputting a third operating element via the touch-sensitive screen, wherein the third operating element comprises a third piece of information relating to at least one of a medical examination via the medical imaging device and contrast-agent administration via the contrast-agent-injection device; and
inputting the first piece of information via a first input command using the first operating element and the third operating element.

3. The method of claim 2, wherein the third piece of information is determined based on the second piece of information.

4. The method of claim 3, further comprising:
outputting a fourth operating element via the touch-sensitive screen, wherein the fourth operating element comprises a fourth piece of information relating to at least one of a medical examination via the medical imaging device and contrast-agent administration via the contrast-agent-injection device; and
inputting the second piece of information via a second input command using the second operating element and the fourth operating element.

5. The method as claimed in claim 4, wherein the fourth piece of information is determined based on the first piece of information.

6. The method of claim 1, further comprising:
executing a software application for operating the medical imaging device and the contrast-agent-injection device including the first operating element and the second operating element.

7. The method of claim 4, further comprising:
executing a first software application for operating the medical imaging device including at least one of the first operating element and the fourth operating element; and
executing a second software application for operating the contrast-agent-injection device including at least one of the second operating element and the third operating element.

8. The method as claimed in claim 7, further comprising:
provisioning an interface for the transmission of information between the first software application and the second software application.

9. The method of claim 8, wherein the interface is based on the client-server model.

10. The method of claim 8, wherein
the interface comprises a memory region,
the first software application has at least one of read and write access to the memory region, and
the second software application has at least one of read and write access to the memory region.

11. The method of claim 6, wherein the software application is executed by a data processing unit.

12. The method of claim 7, wherein at least one of the first software application and the second software application is executed by a data processing unit.

13. The method of claim 4, wherein the software application is executed by a data processing unit, the method further comprising:
transmitting data between the data processing unit and the medical imaging device, wherein the data comprises at least one of the first piece of information and the fourth piece of information.

14. The method of claim 13, further comprising:
transmitting data between the data processing unit and the contrast-agent-injection device, wherein the data comprises at least one of the second piece of information and the third piece of information.

15. The method of claim 1, wherein the software application is executed by a data processing unit and wherein a tablet computer comprises at least one of the touch-sensitive screen and the data processing unit.

16. A system for contrast-agent based medical imaging, comprising:
a medical imaging device;
a contrast-agent-injection device;
a touch-sensitive screen; and
a data processing unit, the data processing unit being embodied to
output a first operating element for operating the medical imaging device via the touch-sensitive screen,
input a first piece of information for operating the medical imaging device via the touch-sensitive screen using the first operating element,
output a second operating element for operating the contrast-agent-injection device via the touch-sensitive screen, and
input a second piece of information for operating the contrast-agent-injection device via the touch-sensitive screen using the second operating element.

17. The system of claim 16, further comprising:
a tablet computer, the tablet computer comprising at least one of the touch-sensitive screen and the data processing unit.

18. A non-transitory computer program product including a computer program, loadable into a memory facility of a data processing unit, including program sections for carrying out the method of claim 1 when the computer program is executed by the data processing unit.

19. A computer-readable medium storing program sections, readable and executable by a data processing unit, for carrying out the method of claim 1 when the program sections are executed by the data processing unit.

20. The method of claim 1, further comprising:
outputting a fourth operating element via the touch-sensitive screen, wherein the fourth operating element comprises a fourth piece of information relating to at least one of a medical examination via the medical imaging device and contrast-agent administration via the contrast-agent-injection device; and
inputting the second piece of information via a second input command using the second operating element and the fourth operating element.

21. The method as claimed in claim 20, wherein the fourth piece of information is determined based on the first piece of information.

22. The method of claim 9, wherein
the interface comprises a memory region,
the first software application has at least one of read and write access to the memory region, and
the second software application has at least one of read and write access to the memory region.
